(19) United States
(12) Patent Application Publication
(10) Pub. No.: US 2008/0275251 A1
(43) Pub. Date: Nov. 6, 2008

(54) Novel Intermediates and Their Use

(75) Inventor: Peng Liu, King of Prussia, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(73) Assignee: Glaxo Group Limited, Greenford, Middlesex (GB)

(21) Appl. No.: 12/093,745

(22) PCT Filed: Nov. 18, 2006
(86) PCT No.: PCT/US06/61065

§ 371 Date: May 15, 2008

Related U.S. Application Data
(60) Provisional application No. 60/737,867, filed on Nov. 18, 2005.

(51) Int. Cl. C07D 233/61

(52) U.S. Cl. 548/334.1

(57) ABSTRACT

The invention is directed to novel acyloxy imidazole intermediates useful for making certain C-14 oxycarbonyl carbamate pleuromutilin derivatives. The invention is further directed to a process for making such acyloxy imidazole intermediates and to a process for making C-14 oxycarbonyl carbamate pleuromutilin derivatives using such acyloxy imidazole intermediates.

### FIELD OF THE INVENTION

The invention is directed to novel acyloxy imidazole intermediates useful for making certain C-14 oxycarbonyl carbamate pleuromutilin derivatives. The invention is further directed to a process for making such acyloxy imidazole intermediates and to a process for making C-14 oxycarbonyl carbamate pleuromutilin derivatives using such acyloxy imidazole intermediates.

### BACKGROUND OF THE INVENTION

International Application No. PCT/EP01/11603, published as International Publication No. WO 02/30929, discloses certain pleuromutilin derivatives useful as antibacterial agents. Specifically, WO 02/30929 discloses C-14 oxycarbonyl carbamate pleuromutilin derivatives according to Formula IA or Formula IB therein.

Certain C-14 oxycarbonyl carbamate pleuromutilin derivatives disclosed therein have exhibited good in vitro and in vivo activity against representative Gram-positive and Gram-negative pathogens associated with respiratory tract and skin and skin structure infections. Accordingly, these derivatives are useful in the treatment of respiratory tract and skin and skin structure infections.

As described in WO 02/30929, the C-14 oxycarbonyl carbamate pleuromutilin derivatives disclosed therein have generally been made through an epimutilin chloroformate intermediate. Synthesis of such derivatives is somewhat lengthy and expensive. In addition, the synthesis involves the use of silver cyanate and phosgene derivatives, both of which present significant difficulties in the large scale production of pharmaceutical products.

In view of the desirable properties C-14 oxycarbonyl carbamate pleuromutilin derivatives exhibit and the shortcomings associated with the known method for preparing such derivatives, there is a need for a process for preparing C-14 oxycarbonyl carbamate pleuromutilin derivatives that is less expensive, uses non-toxic reagents, and proceeds in high yields and in high purities.

### SUMMARY OF THE INVENTION

The invention is directed to novel acyloxy imidazole intermediates useful for making certain C-14 oxycarbonyl carbamate pleuromutilin derivatives. The invention is further directed to a process for making such acyloxy imidazole intermediates and to a process for making C-14 oxycarbonyl carbamate pleuromutilin derivatives using such acyloxy imidazole intermediates.

### DETAILED DESCRIPTION OF THE INVENTION

In describing the invention, chemical elements are identified in accordance with the Periodic Table of the Elements. Abbreviations and symbols utilized herein are in accordance with the common usage of such abbreviations and symbols by those skilled in the chemical and biological arts. For example, the following abbreviations are used herein:

“g” is an abbreviation for grams

“mL” is an abbreviation for milliliters

“° C.” is an abbreviation for degrees Celsius

“DMF” is an abbreviation for the solvent N,N-dimethylformamide

“DSC” is an abbreviation for Differential Scanning Calorimetry

“vol” or “vols” refers to is an abbreviation for volume or volumes, respectively, and refers to the amount of solvent used relative the weight of a starting material. One volume of solvent is defined as 1 mL of solvent for every 1 g of starting material.

“eq” is an abbreviation for molar equivalents

“THF” is an abbreviation for the solvent tetrahydrofuran

“L” is an abbreviation for liters

“N” is an abbreviation for Normal and refers to the number of equivalents of reagent per liter of solution.

“mmol” is an abbreviation for millimole or millimolar

“mol” is an abbreviation for mole or molar

“LOD” is an abbreviation for Loss on Drying

“HPLC” is an abbreviation for High Pressure Liquid Chromatography

“NMR” is an abbreviation of Nuclear Magnetic Resonance

“TLC” is an abbreviation for Thin Layer Chromatography

“LCMS” is an abbreviation for Liquid Chromatography/Mass Spectroscopy

“KF” is an abbreviation for Karl Fischer water determination

“JLR” is an abbreviation for Jacketed Lab Reactor

“TG” and “TGA” are abbreviations for ThermoGravimetric Analysis

“IPA” is an abbreviation for isopropanol, and is also known as 2-propanol

“NMP” is an abbreviation for N-methylpyrrolidinone

“ppm” is an abbreviation for parts per million

### DEFINITIONS

“Alkyl” refers to a saturated hydrocarbon chain having from 1 to 12 member atoms unless otherwise specified. For example, C1-C6 alkyl refers to an alkyl group having from 1 to 6 member atoms. Alkyl groups may be optionally substituted with one or more substituent. Alkyl groups may be straight or branched. Representative branched alkyl groups have one, two, or three branches. Alkyl includes methyl, ethyl, propyl (n-propyl and isopropyl), butyl (n-butyl, isobutyl, and t-butyl), pentyl (n-pentyl, isopentyl, and neopentyl), and hexyl.

“Alkenyl” refers to an unsaturated hydrocarbon chain having from 2 to 12 member atoms unless otherwise specified and having one or more carbon-carbon double bond within the chain. For example, C2-C6 alkenyl refers to an alkenyl group having from 2 to 6 member atoms. In certain embodiments alkenyl groups have one carbon-carbon double bond within the chain. In other embodiments, alkenyl groups have more than one carbon-carbon double bond within the chain. Alkenyl groups may be optionally substituted with one or more substituent. Alkenyl groups may be straight or branched. Representative branched alkenyl groups have one, two, or three branches. Alkenyl includes ethylenyl, propenyl, butenyl, pentenyl, and hexenyl.

“Aryl” refers to an aromatic hydrocarbon ring. Aryl groups are groups are monocyclic ring systems or bicyclic ring systems. Monocyclic aryl ring refers to phenyl. Bicyclic aryl rings refer to napthyl and to rings wherein phenyl is fused to a cycloalkyl or cycloalkenyl ring having 5, 6, or 7 member atoms. Aryl groups may be optionally substituted with one or more substituent.

“Cycloalkyl” refers to a saturated hydrocarbon ring having from 3 to 7 member atoms unless otherwise specified. Cycloalkyl groups are monocyclic ring systems. For example, C3-C6 cycloalkyl refers to a cycloalkyl group having from 3 to 6 member atoms. Cycloalkyl groups may be optionally substituted with one or more substituent. Cycloalkyl includes cyclopropyl, cyclobutyl, cyclopentyl, and cyclohexyl.

“Cycloalkenyl” refers to an unsaturated hydrocarbon ring having from 5 to 7 member atoms unless otherwise specified and having a carbon-carbon double bond within the ring. For example, C5-C6 cycloalkenyl refers to a cycloalkenyl group having from 5 to 6 member atoms. In certain embodiments cycloalkenyl groups have one carbon-carbon double bond within the ring. In other embodiments, cycloalkenyl groups have more than one carbon-carbon double bond within the ring. However, cycloalkenyl rings arc not aromatic. Cycloalkenyl groups arc monocyclic ring systems. Cycloalkenyl groups may be optionally substituted with one or more substituent. Cycloalkenyl includes cyclopentenyl and cyclohexenyl.

“Halo” refers to the halogen radicals fluoro, chloro, bromo, and iodo.

“Heteroaryl” refers to an aromatic ring containing from 1 to 4 heteroatoms as member atoms in the ring. Heteroaryl groups containing more than one heteroatom may contain different heteroatoms. Heteroaryl groups may be optionally substituted with one or more substituent. Heteroaryl groups are monocyclic ring systems or are fused, spiro, or bridged bicyclic ring systems. Monocyclic heteroaryl rings have from 5 to 7 member atoms. Bicyclic heteroaryl rings have from 7 to 11 member atoms. Bicyclic heteroaryl rings include those rings wherein phenyl and a monocyclic heterocycloalkyl ring are attached forming a fused, spiro, or bridged bicyclic ring system, and those rings wherein a monocyclic heteroaryl ring and a monocyclic cycloalkyl, cycloalkenyl, heterocycloalkyl, or heteroaryl ring are attached forming a fused, spiro, or bridged bicyclic ring system. Heteroaryl includes pyrrolyl, pyrrazolyl, imidazolyl, oxazolyl, isoxazolyl, oxadiazolyl, thiazolyl, isothiazolyl, thiadiazolyl, furanyl, furazanyl, thienyl, triazolyl, pyridinyl, pyrimidinyl, pyridazinyl, pyrazinyl, triazinyl, tetrazinyl, tetrazolyl, indolyl, isoindolyl, indolizinyl, indazolyl, purinyl, quinolinyl, isoquinolinyl, quinoxalinyl, quinazolinyl, pteridinyl, cinnolinyl, benzimidazolyl, benzopyranyl, benzoxazolyl, benzisoxazolyl, benzofuranyl, isobenzofuranyl, benzothiazolyl, benzisothiazolyl, benzothienyl, furopyridinyl, and napthyridinyl.

“Heteroatom” refers to a nitrogen, sulphur, or oxygen atom.

“Heterocycloalkyl” refers to a saturated or unsaturated ring containing from 1 to 4 heteroatoms as member atoms in the ring. However, heterocycloalkyl rings are not aromatic. Heterocycloalkyl groups containing more than one heteroatom may contain different heteroatoms. Heterocycloalkyl groups may be optionally substituted with one or more substituent. Heterocycloalkyl groups are monocyclic ring systems or are fused, spiro, or bridged bicyclic ring systems. Monocyclic heterocycloalkyl rings have from 5 to 7 member atoms. Bicyclic heterocycloalkyl rings have from 7 to 11 member atoms. In certain embodiments, heterocycloalkyl is saturated. In other embodiments, heterocycloalkyl is unsaturated but not aromatic. Heterocycloalkyl includes pyrrolidinyl, tetrahydrofuranyl, dihydrofuranyl, pyranyl, tetrahydropyranyl, dihydropyranyl, tetrahydrothienyl, pyrazolidinyl, oxazolidinyl, thiazolidinyl, piperidinyl, homopiperidinyl, piperazinyl, morpholinyl, thiamorpholinyl, azepinyl, 1,3-dioxolanyl, 1,3-dioxanyl, 1,4-dioxanyl, 1,3-oxathiolanyl, 1,3-oxathianyl, 1,3-dithianyl, azetidinyl, azabicylo[3.2.1]octyl, azabicylo[3.3.1]nonyl, azabicylo[4.3.0]nonyl, and oxabicylo[2.2.1]heptyl.

“Member atoms” refers to the atom or atoms that form a chain or ring. Where more than one member atom is present in a chain and within a ring, each member atom is covalently bound to an adjacent member atom in the chain or ring. Atoms that make up a substituent group on a chain or ring are not member atoms in the chain or ring.

“Optionally substituted” indicates that a group, such as alkyl, alkenyl, alkynyl, aryl, cycloalkyl, cycloalkenyl, heterocycloalkyl, or heteroaryl, may be unsubstituted, or the group may be substituted with one or more substituent.

“Pharmaceutically acceptable” refers to those compounds, materials, compositions, and dosage forms which are, within the scope of sound medical judgment, suitable for use in contact with the tissues of human beings and animals without excessive toxicity, irritation, or other problem or complication, commensurate with a reasonable benefit/risk ratio.

“Substituted” in reference to a group indicates that one or more hydrogen atom attached to a member atom within the group is replaced with a substituent. It should be understood that the term “substituted” includes the implicit provision that such substitution be in accordance with the permitted valence of the substituted atom and the substituent and that the substitution results in a stable compound (i.e. one that does not spontaneously undergo transformation such as by rearrangement, cyclization, or elimination and that is sufficiently robust to survive isolation from a reaction mixture). When it is stated that a group may contain one or more substituent, one or more (as appropriate) member atom within the group may be substituted. In addition, a single member atom within the group may be substituted with more than one substituent as long as such substitution is in accordance with the permitted valence of the atom.

### Novel Acyloxy Imidazole Intermediates

The invention is directed, in part, to acyloxy imidazole intermediates having the following general structure:

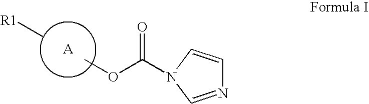

wherein:

A is C4-C6 cycloalkyl, and

R1 is a protected amine or an amine precursor.

The meaning of any functional group or substituent thereon at any one occurrence in Formula I, or any subformula thereof, is independent of its meaning, or any other functional group's or substituent's meaning, at any other occurrence, unless stated otherwise.

As defined above, A is C4-C6 cycloalkyl. In one embodiment of Formula I, A is cyclobutyl. In another embodiment, A is cyclopentyl. In yet another embodiment, A is cyclohexyl. In another embodiment of Formula I, A is otherwise unsubstituted (i.e other than with R1).

As defined above, R1 is a protected amino or an amine precursor.

“Protected amine” refers to a nitrogen atom substituted with one or two suitable protecting groups. “Suitable protecting group” refers to any functional group suitable for protecting the nitrogen atom to which it is attached from reacting with other species in a reaction mixture. Suitable protecting groups and methods for protecting and de-protecting amino groups using such suitable protecting groups are well known to those skilled in the art. Examples of suitable protecting groups may be found in T. Greene and P. Wuts, Protecting Groups in Chemical Synthesis (3rd ed.), John Wiley & Sons, NY (1999).

In one embodiment of Formula I, a suitable protecting group is —C(O)Z1 wherein Z1 is O-alkyl, O-alkenyl, O-cycloalkyl, O-cycloalkenyl, O-heterocycloalkyl, O-aryl, or O-heteroaryl.

In another embodiment of Formula I, a suitable protecting group is —C(O)Z2 wherein Z2 is H, alkyl, alkenyl, cycloalkyl, cycloalkenyl, heterocycloalkyl, aryl, or heteroaryl.

In another embodiment of Formula I, a suitable protecting group is —S(O₂)X1 wherein X1 is alkyl, alkenyl, cycloalkyl, cycloalkenyl, heterocycloalkyl, aryl, or heteroaryl.

In another embodiment of Formula I, a suitable protecting group is —S(O)X2 wherein X2 is alkyl, alkenyl, cycloalkyl, cycloalkenyl, heterocycloalkyl, aryl, or heteroaryl.

In another embodiment of Formula I, R1 is —N(Rx)(Ry) wherein Rx and Ry taken together with the nitrogen atom to which they are attached form an optionally substituted succinimide ring, an optionally substituted maleimide ring, or an optionally substituted phthalimide ring.

In another embodiment of Formula I, R1 is —NC(Ra)(Rb) wherein Ra is H, alkyl, alkenyl, cycloalkyl, cycloalkenyl, heterocycloalkyl, aryl, or heteroaryl; and Rb is alkyl, alkenyl, cycloalkyl, cycloalkenyl, heterocycloalkyl, aryl, or heteroaryl.

In another embodiment, R1 is —NC(H)N(Rc)(Rd) wherein Rc is alkyl, alkenyl, cycloalkyl, cycloalkenyl, heterocycloalkyl, aryl, or heteroaryl; and Rd is alkyl, alkenyl, cycloalkyl, cycloalkenyl, heterocycloalkyl, aryl, or heteroaryl.

Representative suitable protecting groups include: tert-butoxycarbonyl, benzyloxycarbonyl, trichloroethoxycarbonyl, 9-fluorenylmethyloxycarbonyl, allyloxycarbonyl, cinnamyloxycarbonyl, formyl, acetyl, trifluoroacetyl, trichloroacetyl, benzoyl, substituted benzoyl, phthalimide, succinimide, allyl, benzyl, substituted benzyl groups such as methoxy benzyl, diphenyl methyl and triphenyl methyl, aldimines such as N-benzylidene, ketimines such as benzophenone imine, N,N-dialkyl or diaryl amidines, and sulfonamides such as p-toluenesulfonamide.

“Amine precursor” refers to any functional group capable of being converted to an amine.

In one embodiment of Formula I, R1 is —C(O)R3 wherein R3 is —OH, —NH₂, —N₃, —N(H)NH₂, or —N(H)OC(O)Re wherein Re is H, alkyl, alkenyl, cycloalkyl, cycloalkenyl, heterocycloalkyl, aryl, or heteroaryl.

In another embodiment of Formula I, R1 is —NO₂.

In another embodiment of Formula I, R1 is ═O.

### Compounds Prepared Using the Present Invention

The acyloxy imidazole intermediates according to Formula I above are useful for making certain C-14 oxycarbonyl carbamate pleuromutilin derivatives. Thus, the invention is further directed to a process for making C-14 oxycarbonyl carbamate pleuromutilin derivatives having the following general structure:

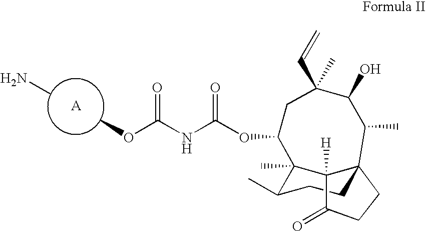

wherein:

A is C4-C6 cycloalkyl, or a pharmaceutically acceptable salt thereof.

As defined above, A is C4-C6 cycloalkyl. In one embodiment of Formula II, A is cyclobutyl. In another embodiment, A is cyclopentyl. In yet another embodiment, A is cyclohexyl. In another embodiment of Formula II, A is otherwise unsubstituted (i.e other than with NH₂).

### Process for Making the Novel Acyloxy Imidazole Intermediates

The process for making acyloxy imidazole intermediates according to Formula I above begins with providing a compound having the following general structure:

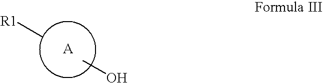

wherein R1 and A are as defined above for Formula I.

Compounds according to Formula III are made from commercially available starting materials using methods known to the skilled artisan. For example, compounds according to Formula III wherein A is cyclohexyl and R1 is a protected amine can be made from commercially available trans-4-aminocyclohexanol. The trans-4-aminocyclohexanol is dissolved in a suitable solvent and this is followed by the addition of a suitable protecting group agent to generate a compound according to Formula III. Suitable solvents for the reaction include N-methyl pyrrolidinone (NMP), N,N-dimethyl formamide (DMF), acetonitrile, dimethylsulfoxide, tetrahydrofuran, ethyl acetate and 1,2-dimethyoxyethane. In one embodiment of the invention, the trans-4-aminocyclohexanol is dissolved in NMP or DMF. A suitable temperature range for this reaction is between about 20° C. and about 120° C. In one embodiment of the invention, this reaction is carried out at a temperature from about 50° C. to about 60° C.

The next step in the process is reacting the compound according to Formula III with 1,1′-carbonyldiimidazole to yield a compound according to Formula I. The compound according to Formula III may be isolated or it may be reacted in situ with 1,1′-carbonyldiimidazole to yield the compound according to Formula 1. 1,1′-carbonyldiimidazole is commercially available. A suitable temperature range for this reaction is between about 0° C. and about 70° C. In one embodiment of the invention, this reaction is carried out at a temperature from about 20° C. to about 30° C.

The compound according to Formula I may optionally be isolated. Compounds according to Formula I can be isolated by methods known to those skilled in the art. Such methods include extraction, solvent evaporation, and crystallization.

### Process for Making the C-14 Oxycarbonyl Carbamate Pleuromutilins

The process for making the C-14 oxycarbonyl carbamate pleuromutilin derivatives according to Formula II above begins with providing the compound having the following general structure:

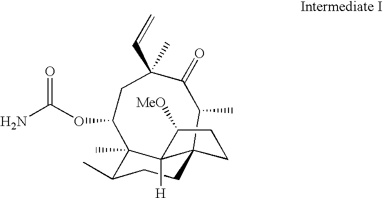

Generally speaking, Intermediate I may be prepared from pleuromutilin or from mutilin. Pleuromutilin may be produced by the fermentation of microorganisms such as Clitopilus species, Octojuga species and Psathyrella species using methods known to those skilled in the art. The pleuromutilin is then typically isolated from the fermentation broth with organic solvent. Pleuromutilin may be converted to mutilin by alkaline hydrolysis. Such methods are well known in the art. The preparation of Intermediate I is described below in Examples 1, 2, and 3. Other starting materials and reagents are commercially available or are made from commercially available starting materials using methods known to those skilled in the art.

The next step in the process is reacting Intermediate 1 with a base in a suitable solvent to form the anion of Intermediate 1. Suitable bases include alkoxide bases, such as the lithium, sodium, and potassium salts of isopropanol, tert-butanol, sec-butanol, and tert-pentanol, lithium bis(trimethylsilylamide), sodium bis(trimethylsilylamide, potassium bis(trimethylsilylamide), lithium diisopropylamide, and lithium dicyclohexylamide. In one embodiment of the invention, the base is sodium tert-pentoxide or lithium bis(trimethylsilylamide). Suitable solvents include tetrahydrofuran (THF), toluene, acetonitrile, N,N-dimethylformamide (DMF), N-methylpyrollidinone (NMP), tert-butyl methyl ether, dichloromethane. In one embodiment of the invention, the solvent is tert-butyl methyl ether, acetonitrile or NMP. A suitable temperature range for this reaction is between −10° C. and 30° C. In one embodiment of the invention, the temperature range for this reaction is between −5° C. and 10° C.

The next step in the process is reacting the anion of Intermediate 1 with a compound according to Formula 1 to produce a compound having the following general structure:

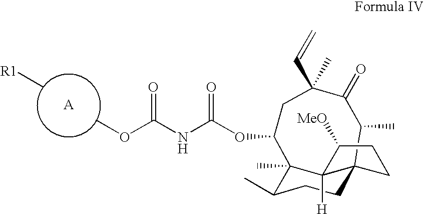

A suitable temperature range for this reaction is between −10° C. and 30° C. In one embodiment of the invention, the temperature range for this reaction is between −5° C. and 10° C.

The next step is modifying the compound according to Formula IV to form a compound according to Formula II. The compound according to Formula IV may be isolated prior to modification to form a compound according to Formula II. Methods for isolation are known to those skilled in the art and include extraction, solvent evaporation, and crystallization. For example, the compound according to Formula IV can be isolated by crystallization, induced by addition of an antisolvent, such as water or water containing a small amount of an acid, such as ammonium chloride, acetic acid, or HCl. Alternatively, the compound according to Formula IV may be reacted further without isolation to produce a compound according to Formula II.

Compounds according to Formula Iv wherein R1 is a protected amine and the suitable protecting group(s) is acid labile can be modified to form a compound according to Formula II by reacting the compound according Formula IV with a strong acid in the presence of water. Suitable strong acids include HCl, H3PO4, H2SO4, trifluoracetic acid, methanesulfonic acid, and toluenesulfonic acid. In one embodiment of the invention, the acid is HCl. This reaction may optionally be done in the presence of organic solvents, such as tert-butyl methyl ether, ethyl acetate, acetonitrile, toluene, tetrahydrofuran and dichloromethane. In one embodiment of the invention, the solvent is tert-butyl methyl ether or ethyl acetate. A suitable temperature range for this reaction is between 20° C. and 50° C. In one embodiment of the invention, the temperature range for this reaction is between 35° C. and 45° C.

Compounds according to Formula IV wherein R1 is a protected amine and the suitable protecting group(s) is not acid labile, can be modified to form a compound according to Formula II by deprotecting the protected amine and converting the mutilin core to the pleuromutilin core. Deprotection of the protected amine can be accomplished using methods known to those skilled in the art, such as those methods taught in Greene and Wuts. Deprotection of the protected amine produces a compound having the following general structure:

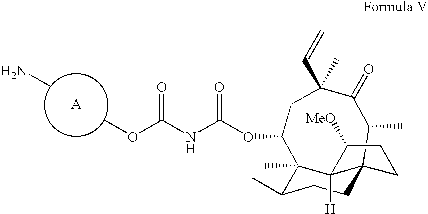

Conversion of compounds according to Formula V to a compound according to Formula II can be accomplished by reacting a compound of Formula V with a strong acid in the presence of water.

Compounds according to Formula IV wherein R1 is an amine precursor can be modified to form a compound according to Formula II by converting the amine precursor to an amine and converting the mutilin core to the pleuromutilin core. Conversion of the amine precursor to an amine can be accomplished using methods known to those skilled in the art. For example, rearrangement of carboxylic acids (where R1 is C(O)OH) can be accomplished by a Schmidt reaction; rearrangement of carboxamides (where R1 is C(O)NH2) can be accomplished by a Hofmann rearrangement; rearrangement of acyl azides (where R1 is C(O)N3) can be accomplished by a Curtius rearrangement; and rearrangement of O-acyl hydroxamic acids (where R1 is C(O)NHOC(O)Z2) can be accomplished by a Lossen rearrangement (and its derivatives) to produce an amine. Such methods are known to those skilled in the art. Compounds according to Formula IV wherein R1 is —NO₂, can be converted to a compound according to Formula V by reduction of the nitro group, which can be accomplished using methods known to those skilled in the art. Compounds according to Formula IV wherein R1 is ═O, can be converted to a compound according to Formula V by reductive amination of the oxo group, which can be accomplished using methods known to those skilled in the art. Operation of any of these conversions on a compound according to Formula IV wherein R1 is an amine precursor will produce a compound according to Formula V, which can then converted to a compound according to Formula II by treatment with a strong acid in the presence of water.

As indicated above, acidic conditions are required to convert the mutilin core to the pleuromutilin core to produce a compound according to Formula II. At this point, the compound of Formula II can be isolated as the HCl salt or as the free base by methods known to those skilled in the art. Such methods include extraction, solvent evaporation, and crystallization.

Thus, in one embodiment of the invention, the crystallization of the compound according to Formula II produces it as the HCl salt form. In this embodiment, the pH of the acidic reaction mixture containing Formula II, described above, can be adjusted to pH 3.0 to pH 5.5 using a base to induce crystallization. A suitable pH range is pH 4.5 to pH 5.0. Bases such as ammonium hydroxide, sodium hydroxide, lithium hydroxide, potassium hydroxide, potassium carbonate, sodium carbonate, ammonium carbonate, sodium bicarbonate, potassium bicarbonate, sodium phosphate, and sodium acetate can be employed, especially ammonium hydroxide. A suitable temperature range for crystallization is between −10° C. and 30° C. In a further embodiment of the invention, the temperature range for this reaction is between −5° C. and 20° C.

In another embodiment, the isolation of the compound according to Formula II produces it as the free base form. In this embodiment, the pH of the acidic reaction mixture containing the compound according to Formula II, described above, can be adjusted to pH 7.0 to pH 10.0 using a base followed by extraction into an organic solvent and crystallization. A suitable pH range is pH 9.0 to pH 9.5. Bases such as ammonium hydroxide, sodium hydroxide, lithium hydroxide, potassium hydroxide, potassium carbonate, sodium carbonate, ammonium carbonate, sodium bicarbonate, potassium bicarbonate, sodium phosphate, and sodium acetate can be employed, especially sodium or potassium carbonate. Suitable organic solvents include dichloromethane, ethyl acetate, isopropyl acetate, methyl isobutyl ketone, and dimethyl carbonate, especially methyl isobutyl ketone. Crystallization can be induced by methods known to those skilled in the art including cooling, concentrating or adding an anti-solvent to the solution to induce a state of supersaturation. Suitable anti-solvents include 1-propanol and 2-propanol.

Alternative salts of the compound according to Formula II can then be obtained by dissolving the free base of the compound according to Formula II in a suitable solvent and adding an acid. Suitable solvents include methanol, 1-propanol and 2-propanol. Suitable acids include succinic acid and 1,2-ethanedisulfonic acid.

Alternative salts of the compound according to Formula II can also be obtained by dissolving the HCl salt of the compound according to Formula II in a suitable solvent or solvent mixture and neutralizing the HCl by addition of an aqueous base. Upon removing the aqueous component of the mixture, alternative salts of the compound according to Formula II can be obtained by adding a suitable acid to the solution of the free base of Formula II. Suitable organic solvents include dichloromethane, ethyl acetate, isopropyl acetate, methyl isobutyl ketone, 1-propanol, 2-propanol and dimethyl carbonate, or mixtures thereof. Suitable acids include succinic acid and 1,2-ethanedisulfonic acid.

### EXAMPLES

The following examples illustrate the invention. These examples are not intended to limit the scope of the present invention, but rather to provide guidance to the skilled artisan to prepare and the compounds of the invention.

### Example 1

### Preparation of Intermediate 1

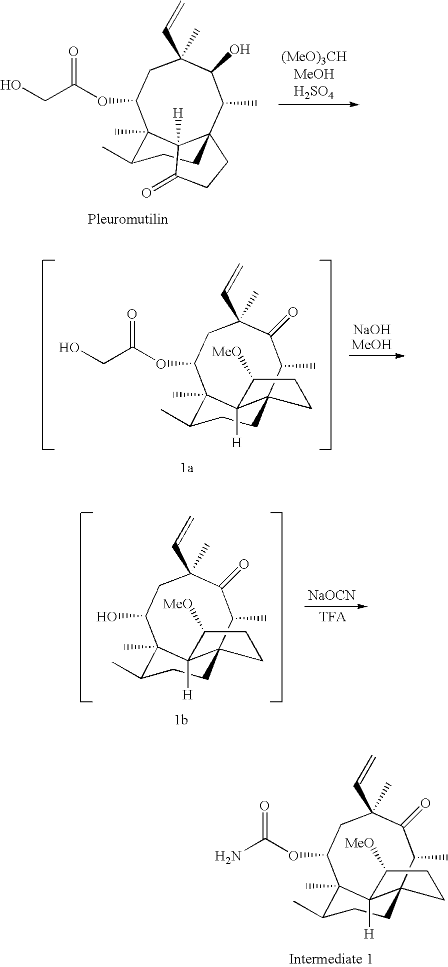

To a reaction vessel under nitrogen atmosphere were charged pleuromutilin (59.2 grams), methanol (240 mL) and trimethyl orthoformate (95 mL). The mixture was cooled to 0° C. Concentrated sulfuric acid (18 mL) was added slowly to keep the reaction temperature below 10° C. After addition, the reaction mixture was heated to 30° C. After 3 hours at 30° C. and 14 hours at 18° C., the reaction was deemed complete by HPLC analysis. The crude product in the reaction mixture was used in next reaction directly.

1a in the reaction mixture was cooled to −10° C. Water (70 mL) was added slowly to keep the internal temperature below 15° C. An aqueous solution of sodium hydroxide (135 mL, 50% w/w) was charged slowly to keep the internal temperature below 15° C. The reaction was then heated to 65° C. After 30 minutes at 65° C. the reaction was complete based on HPLC analysis. The reaction was cooled to ˜40° C. Methanol was distilled out under reduced pressure. Water (300 mL) and toluene (350 mL) were added to the mixture. The mixture was heated to ˜65° C. and was stirred for 10 minutes. After settling for 30 minutes, the aqueous layer was separated. The aqueous layer was extracted with toluene (200 ml). The organic layers were combined and distilled under reduced pressure to a final volume of ˜300 mL. The crude product in toluene was used directly in next reaction.

To the product from above in toluene was added more toluene (350 mL) at ambient temperature. Sodium cyanate (27.4 grams) was added with stirring. Trifluoroacetic acid (29 mL) was slowly added over 0.5 hour. The mixture was stirred for 14 hours at ambient temperature. No starting material was detected in the reaction mixture by HPLC analysis. Water (360 mL) was added to the reaction with stirring. The layers were separated and the aqueous layer was discarded. Toluene was distilled wider reduced pressure until a final volume of ˜100 mL. Heptane (300 mL) was added. The mixture was stirred at 65° C. for 30 minutes then cooled to 0° C. and stirred for one hour. The resulting slurry was filtered and washed twice with cold heptane (80 mL). The crude product was dried at 65-70° C. under vacuum to give 42.1 grams of Intermediate 1. Yield: 71%.

### Example 2

### Preparation of Intermediate 1

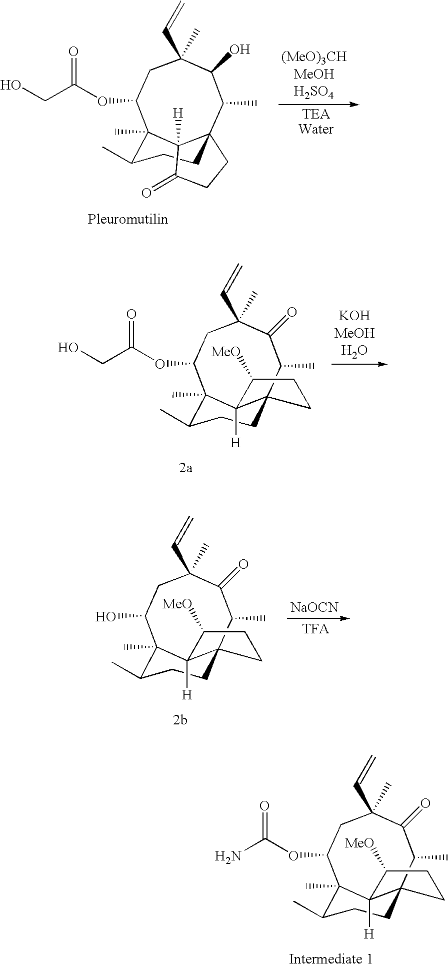

To a reaction vessel under nitrogen atmosphere were charged pleuromutilin (20.0 grams), methanol (80 mL) and trimethyl orthoformate (32 mL). The mixture was cooled to 0° C. Concentrated sulfuric acid (6 mL) was added slowly to keep the reaction temperature below 10° C. After addition, the reaction mixture was heated to 30° C. After 5 hours at 30° C. and 14 hours at 18° C., the reaction was deemed completed by HPLC analysis. The reaction mixture was cooled to ˜10° C. Triethylamine (32 mL) was added slowly to keep the internal temperature below 30° C. Water (110 mL) was added to the reaction with vigorous stirring. The mixture was stirred at ˜20° C. for 4 hours. The crude product was filtered and washed with water (60 mL) twice. The wet solid was dried at 50° C. under vacuum to give 16.0 grams of product. Yield: 77%.

To a flask were charged methanol (80 mL) and water (10 mL). Potassium hydroxide (5.7 g) was added. The mixture was stirred for ˜5 minutes to a solution. 2a (20.0 g) was added to the mixture. The reaction mixture was heated to 65° C. and stirred for 1 hour. The reaction was deemed complete by HPLC analysis and the mixture was cooled to ˜25° C. and slowly transferred into a larger flask containing water (100 mL) and 2b seed (50 mg) with vigorous stirring. The resulted slurry was cooled to ˜5° C. and stirred for 1 hour. The crude 2b was filtered and washed with water (50 mL) twice. The wet product was dried at ˜65° C. for 24 hours to give 15.3 grams of solid. Yield: 90%.

To a flask were charged toluene (180 mL), 2b (20.0 g) and sodium cyanate with stirring. Trifluoroacetic acid (10 mL) was slowly added over 1 hour. The mixture was stirred for 16 hours at ambient temperature after which no 2b was detected by HPLC analysis. Water (100 mL) was added to reaction with stirring and the layers were separated. The aqueous layer was discarded and the toluene layer was concentrated under reduced pressure to a final volume of ˜30 mL. Heptane (100 mL) was added and the mixture was stirred at 65° C. for 30 minutes. The mixture was cooled to 0° C. and stirred for 1 hour. The resulted slurry was filtered and washed with cold heptane (20 mL, ˜0° C.) twice. The crude product was dried at 65° C. under vacuum to give 19.1 grams of Intermediate 1. Yield: 85%.

### Example 3

### Preparation of Intermediate 1

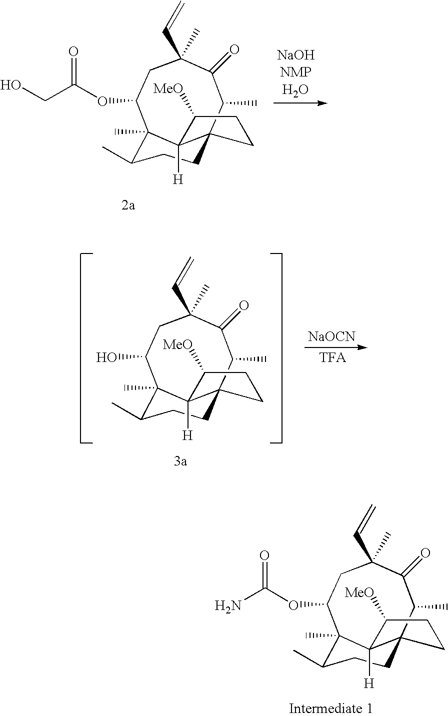

To a flask were charged N-methyl pyrrolidone (24 mL), 2a solid (from Example 2) (12.0 grams), and water (10 mL). Sodium hydroxide aqueous solution (20 mL, 50% w/w) was added. The reaction mixture was heated to 70° C. and stirred for 1 hour. Toluene (120 mL) was added to the mixture, stirred for 30 minutes and the layers were separated. The toluene layer was washed with water (30 mL) and concentrated under vacuum to ˜100 mL final volume. The crude product in toluene was used directly in the next reaction.

To 3a in toluene was added sodium cyanate. Trifluoroacetic acid (5 mL) was slowly added over 1 hour. The mixture was stirred for ˜15 hours at ambient temperature until no 3a was detected by HPLC analysis. Water (30 mL) was added to reaction with stirring, the layers were separated, and the aqueous layer was discarded. Toluene was distilled under reduced pressure until ˜10 mL remained. Heptane (50 mL) was added and the mixture was stirred at 65° C. for 30 minutes. The mixture was cooled to 0° C. and stirred for one hour. The resulting slurry was filtered and was washed twice with cold heptane (15 mL each, ˜0° C.). The crude product was dried at 65° C. under vacuum to give 9.5 grams of Intermediate 1. Yield: 82%.

### Example 4

### Preparation of N-Boc-trans-3-aminocyclobutanol

### Stage 1

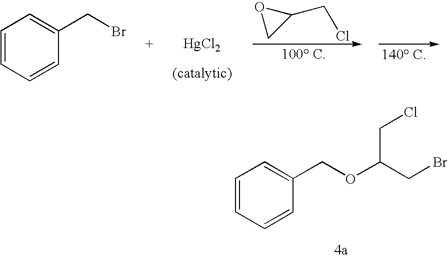

To a solution of mercury chloride (5.3 g) in benzyl bromide (2.31 kg) at 100° C. or reflux was slowly added epichlorohydrin (1.25 kg) over forty minutes. The reaction mixture was then heated to an internal temperature about ˜135° C. for ˜3 hours, cooled to room temperature overnight and heated for an additional ˜12 h at ˜135-150° C. The mixture was cooled to ambient temperature and left overnight. The mixture was then purified via reduced pressure distillation. A yield of 70% 1-Bromo-2-O-benzyl-3-chloropropane (4a, 2.51 kg) was obtained.

### Stage 2

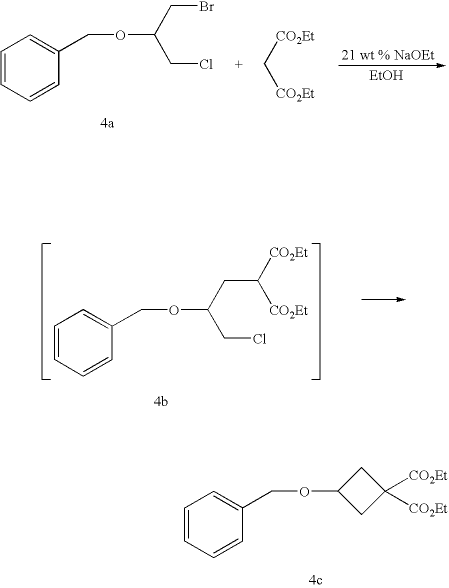

To a solution of 4a (80 g) and diethyl malonate (121.7 g, 2.5 equiv) in EtOH (160 mL) was slowly charged NaOEt (21 wt % in EtOH) (284 mL, 2.5 equiv) through addition funnel. The mixture was heated to reflux (˜80° C. internal temperature) then stirred for additional ˜3 hours before sampling and concluding that 4a was consumed based on HPLC results. The mixture was cooled to ˜35° C. and filtered through filter paper. The filtrate was concentrated by distillation until ˜420 mL of distilled solvent was collected. The mixture was heated to ˜125° C. and stirred for ˜2 hours before sampling and concluding that 4b was consumed based on HPLC results. The mixture was cooled to room temperature. Water (160 mL) and ethyl acetate (320 mL) were charged. The mixture was stirred and two layers were separated. The organic layer was washed with water (80 mL). The organic layer was concentrated under reduced pressure to dryness. The product was dried under vacuum to obtain crude 4c, 125.1 g.

### Stage 3

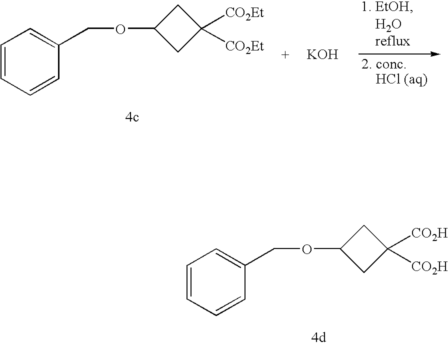

A KOH solution was prepared by adding KOH (2.02 kg, 5 equiv) to water (2.55 L). A 20 L jacketed laboratory reactor was charged with crude 4c (1.7 kg) and EtOH (6.8 L). The KOH solution was charged in 2 portions which caused the internal temperature to rise to 56° C. The mixture was heated over ˜40 minutes until brought to reflux (˜79° C. internal temperature) then stirred for and additional 30 minutes before sampling and concluding that 4c was consumed based on HPLC results. The mixture was cooled slightly then concentrated under reduced pressure until ˜5.2 L of solution remained in the reactor. While continuing to cool the reactor, the contents were diluted with water (5.1 L). When the temperature of the Solution reached ˜16° C., concentrated HCl (aqueous) was slowly added in portions until the aqueous layer pH was adjusted to 2.5-3 (a total of 2.2 L of concentrated HCl was used). Methyl t-butyl ether (8.5 L) was charged. The mixture was stirred and the layers were separated. The organic layer was washed with water (1.7 L). The organic layer was held at ˜20° C. overnight, then concentrated under reduced pressure until ˜2.8 L remained in the reactor. Toluene (8.5 L) was charged and the mixture was concentrated under reduced pressure until ˜6.8 L of distilled solvent was collected. Toluene (6.8 L) was charged and the mixture was heated to ˜90° C. over 50 minutes, then cooled back to 16° C. over 50 minutes. The solid was filtered and rinsed with cyclohexane (1.7 L). The solid was dried under vacuum at 50° C. for ˜2 days and 1.04 kg of dried product 4d was obtained.

### Stage 4

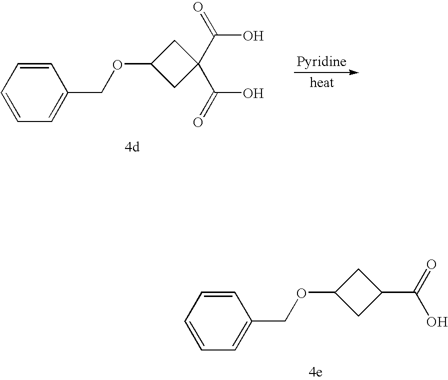

4d (783.35 g) was charged to a 3 L round bottom flask. Pyridine (783 mL) was added. The solution was heated to ˜117° C. for 8-12 hours. The reaction was deemed complete when HPLC monitoring indicated that <2% of 4d remained. The solution was concentrated under vacuum on a rotary evaporator until no additional distillate could be seen. After holding the residue overnight, toluene (4.7 L) was added, followed by slow addition of an HCl solution (1.0 N, 3.13 L) at such a rate that the temperature was maintained <30° C. during the addition. The mixture was stirred for 10 minutes. The two layers were separated and the aqueous layer was extracted with 2.35 L of toluene. The combined organic layers were washed with 783 mL of brine and the layers were again separated. The organic layer was concentrated until ˜2.35 L of solution remained. Further concentration under vacuum provided an oil (619 g). The yield was estimated to be ˜77% based on a weight/weight assay using HPLC.

### Stage 5

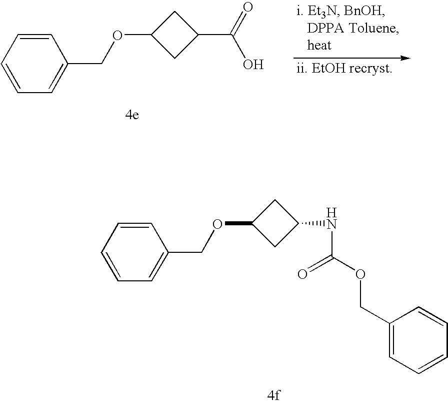

A solution of 4e in toluene (assumed 3.6 kg, 17.5 mmol), triethylamine (3.5 kg, 34.9 mol), and benzylalcohol (1.9 kg, 17.5 mol) in total amount of toluene (36 L) was prepared then heated to an internal temperature between 70 and 80° C. Diphenylphosphorylazide (4.9 kg, 18.0 mol) was slowly added over 35 minutes while maintaining temperature between 70 and 80° C. The vessel containing the diphenylphosphorylazide was rinsed with 1 L toluene and added to reactor. Once the addition was complete, the contents were held for ˜15 minutes at ˜80 C, then the reaction mixture was heated to an internal temperature ˜100° C. and stirred for about 11.5 hours. The reaction mixture was cooled to ˜20° C., and held overnight. The reaction mixture was partially concentrated by vacuum distillation to ˜15 L. Ethyl acetate (EtOAc, 40 L) and 0.25 N aqueous sodium hydroxide solution (18 kg) were added. The layers were separated. The organic layer was washed with 0.25 N NaOH (22.4 kg). EtOAc was removed via vacuum distillation to a minimum stirrable volume (˜18 L). Ethanol (200 proof, 18 L) was added and residual EtOAc was removed via vacuum distillation to ˜18 L. The mixture was heated to 75° C. to dissolve all solids (˜15 min), then cooled to between 30 and 40° C. The mixture was seeded with 0.1 wt % 4f (3.6 g) and slowly cooled to 0° C. at a rate of 10° C. per hour. Ethanol (200 proof, 5 L) was added to maintain a stirrable mixture. The mixture was slurried at 0° C. for ˜13.5 hours. The solids were filtered and the cake was washed with ethanol (200 proof, 7.2 L) then dried at 50° C. under vacuum to provide 4f (1.1 kg, 20.2% yield, 98.1% chemical purity, 97.9% isomeric purity).

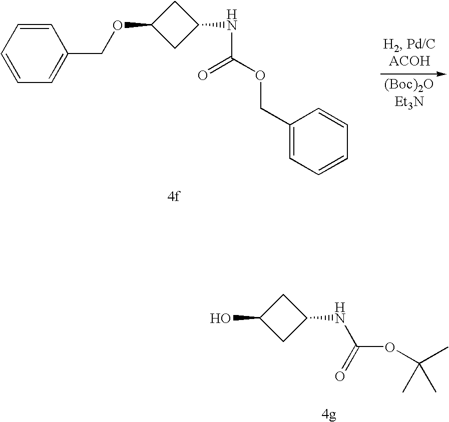

### Stage 6

A suspension of 4f (27 g) and Pd/C (3.9 g, 10% w/w) in acetic acid (163 mL) was shaken under ˜50 psi for 1 hour at 20° C. and heated at 50° C. for ˜3-5 hours. The reaction mixture was cooled to room temperature, filtered, washed with ethanol, and the filtrate concentrated until ˜1 volume was left. The residue oil was dissolved in ethanol (55 mL) and triethylamine (55 mL). Di-t-butoxy dicarbonate (14.5 g) was added, and the reaction mixture was stirred at room temperature over the weekend. The solution was concentrated to minimum volume. Water (110 mL) and methylene chloride (68 mL) and then saturated sodium bicarbonate (34 mL) were added. Two layers were separated. The aqueous layer was extracted with methylene chloride (2×68 mL) again. The combined organic layers were washed with brine (33 mL). The solution was then concentrated. Cyclohexane (108 mL) was added and then concentrated to 3.0 volumes. The solid was filtered, washed with cyclohexane (27 ml) and the wet cake was dried under vacuum at 50° C. to provide 15.5 g of product. 4g in a yield of 95%.

### Example 5

### Preparation of Intermediate 4f from Example 4

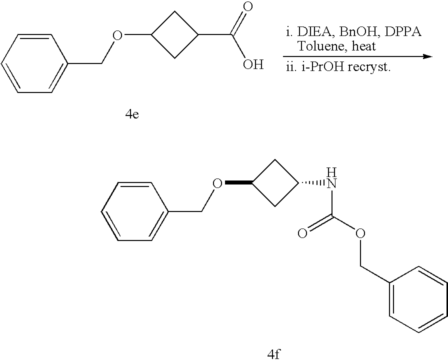

A solution of 4e (22.3 g) in toluene (220 mL) and N,N-diisopropylethylamine (41 mL) was prepared then heated to an internal temperature between 95 and 105° C. Diphenylphosphorylazide (26 mL) was slowly added over 40 minutes at a rate to maintain manageable levels of heat and gas evolution. The mixture was stirred for 10 minutes, then benzyl alcohol (12.5 mL) was added and the reaction was stirred for about 10 hours at a temperature about 95-° C. The reaction mixture was cooled to ambient temperature. Diluted with Ethyl acetate (EtOAc, 250 mL) then washed with 0.25 N aqueous sodium hydroxide (NaOH) solution (125 mL). The layers were separated. The organic layer was washed with 0.25 N NaOH (150 mL). EtOAc was removed via rotary evaporation. Isopropanol (100 mL) was added and the mixture was heated to 80° C. to dissolve all solids, then cooled to between 30 and 40° C. The mixture was seeded with 0.1 wt % 4f at an internal temperature of 35° C. and slowly cooled to between 0° C. and 5° C. The mixture was slurried at ˜0° C. for 1 hour, then filtered and washed with isopropanol (0-10° C.) as needed (˜15 ml). The product was dried at 50° C. under vacuum to provide 4f (13.3 g, 39% yield, 90.6% isomeric purity, 90.2% chemical purity).

### Example 6

### Preparation of N-Boc-trans-4-aminocyclohexanol

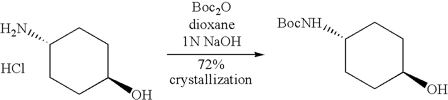

Trans-4-aminocyclohexanol hydrochloride (Aldrich, 96.5 g, 0.636 mole) was added with mechanical stirring to 636 mL of 1.0N NaOH in a 3 L 3N RBF, equipped with a 500 mL addition funnel. After a clear solution was obtained 300 mL of dioxane was added, and then the mixture was cooled in an ice bath to about 5° C. A solution of di-tert-butyldicarbonate (Aldrich, 138.9 g, 0.636 mole) in dioxane (300 mL) was added to the addition funnel and added dropwise to the reaction mixture over 1 hour. The reaction was then allowed to slowly warm to room temperature with stirring in the bath overnight (milky white mixture). The whole was extracted with EtOAc (1×800 mL, 2×500 mL), the organics were combined and washed with water and brine. At this point the product began to precipitate from the organic layer. 400 mL of ethanol was added to aid solubility of the product. The mixture was dried over magnesium sulfate then filtered and evaporated to 500 mL. Then hexanes (600 mL) was added slowly with stirring. The product, N-Boc-trans-4-aminocyclohexanol, was filtered, and washed thoroughly with hexanes to afford the product as white flakes. (98 g, 72%).

### Example 7

### Preparation of trans-3-({[(1,1-dimethylethyl)oxy]carbonyl}amino)cyclobutyl 1H-imidazole-1-carboxylate

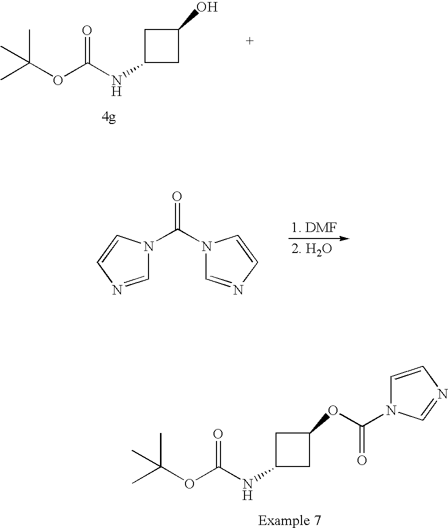

To a solution of 4 g (17.9 g) in N,N-dimethylformamide (DMF) (72 mL) resulting in a clear solution was added 1,1′-carbonyldiimidazole (CDI) (20.2 g, 1.3 equiv) was charged, which caused the internal temperature to rise to ˜30° C. The mixture was stirred at room temperature for ˜30 minutes before sampling and concluding 4 g was consumed based on HPLC results. While the mixture was cooled with ice bath, water (˜160 mL) was added and the mixture was stirred at ˜0° C. for 45 minutes. The solid was filtered and rinsed with water (˜80 mL). The solid was dried under vacuum at ˜55° C. overnight and 24 g of product Example 7 was obtained.

### Example 8

### Preparation of trans-4-({[(1,1-dimethylethyl)oxy]carbonyl}amino)cyclohexyl 1H-imidazole-1-carboxylate

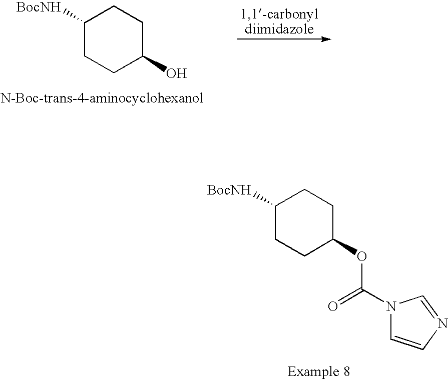

To a 1 L flask was charged N-Boc-trans-4-aminocyclohexanol (made from commercially available trans-4-aminocyclohexanol using methods known to those skilled in the art, 30.4 g, 0.141 mole) and DMF (338 mL). 1,1′-carbonyldiimidazole (“CDI”, 162.15 g, 0.184 mole) was charged in one portion. The mixture was stirred at 18° C. for 2.5 hours until HPLC analysis indicated there was no N-Boc-trans-4-aminocyclohexanol present. The reaction was then cooled to 10° C. Water (450 mL) was added slowly to keep temperature below 20° C. The mixture was cooled to 0° C. and was stirred for 1 hour. The product was filtered and washed with water (50 mL) twice. The wet product was dried at 60-65° C. under vacuum for 24 hours. Yield: 40.1 g, 92%.

### Example 9

### Preparation of trans-4-({[(1,1-dimethylethyl)oxy]carbonyl}amino)cyclohexyl 1H-imidazole-1-carboxylate

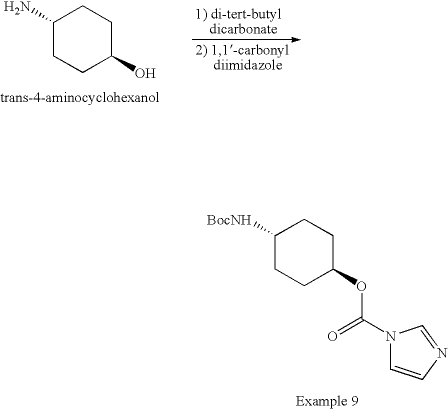

To a 250 ml flask under nitrogen atmosphere was charged DMF (98 mL) and di-t-butyl dicarbonate (63.6 g, 0.291 mole). The mixture was stirred at ambient temperature to give a solution. To a second 1 L flask was charged trans-4-aminocyclohexanol (commercially available) (32.5 g, 0.285 mole) and DMF (160 mL). The mixture was heated to 55-60° C. to give a solution. Approximately 33% of the di-t-butyl dicarbonate in DMF solution was slowly added to the trans-4-aminocyclohexanol over approximately 30 minutes, keeping the process temperature below 75° C. during the charge. The mixture was stirred for ˜30 min-1 hour at 55-60° C. The slow addition of ˜33% of the di-t-butyl dicarbonate DMF solution was repeated twice more and the mixture was stirred for ˜30 min-1 hour at 55-60° C. after each addition. After the last addition the reaction mixture was stirred at 70-75° C. over 1-2 hours. When GC analysis indicated that the ratio between intermediate N-Boc-trans-4-aminocyclohexanol and trans-4-aminocyclohexanol was greater than 33:1, the reaction mixture was cooled to 15-20° C. DMF (250 mL) was charged to the reaction and stirred to give a solution. 1,1′-Carbonyldiimidazole (59.2 g, 1.3 eq) was charged to the reaction in one portion. The reaction was stirred at ˜19° C. for 15 hours and reaction progress was monitored by HPLC. When the HPLC analysis indicated that the ratio between Intermediate 2 and N-Boc-trans-4-aminocyclohexanol was greater than 49:1, the reaction mixture was cooled to ˜0° C. Water (600 mL) was charged slowly to the reaction mixture while keeping the process temperature below ˜20° C. The white solid product precipitated out of solution. The mixture was cooled to ˜0° C. and stirred for 1 hour. The product was filtered and the wet cake was washed with water (100 mL) twice. The product was dried at 60-65° C. under vacuum to a constant weight. Yield: 82.1 g, 94%.

### Example 10

### Preparation of trans-3-aminocyclobutyl (1S,2R,3S,4S,6R,7R,8R,14R)-4-ethenyl-3-hydroxy-2,4,7,14-tetramethyl-9-oxotricyclo[5.4.3.01,8]tetradec-6-yl imidodicarbonate L-tartrate

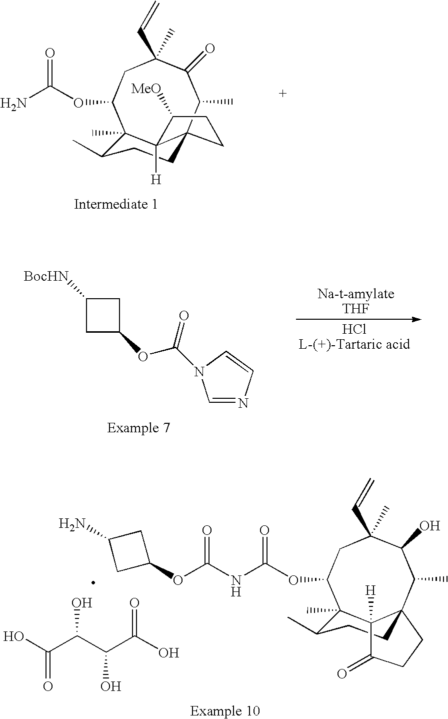

A solution of Intermediate 1 (1.89 g, 5 mmol) in THF (19 mL) was cooled to ˜17° C. Sodium tert-pentoxide (1.38 g, 12.5 mmol, 2.5 eq.) was added in one portion. The solution was stirred for 30 minutes before addition of Example 7 (1.54 g, 5.5 mmol, 1.1 eq.) in one portion at ˜18° C. The solution was stirred for one to two hours. Water (9.5 mL) was added slowly to quench the reaction, followed by the addition of saturated ammonium chloride solution (9.5 mL). Ethyl acetate (17 mL) was added and then the mixture was stirred for 5 minutes. The resulting two layers were separated and the aqueous layer was extracted with ethyl acetate (3.8 mL). The combined organic layers were washed with saturated ammonium chloride solution (1.9 mL) then concentrated to ˜15 mL.

Hydrochloric acid (cone. 8.0 mL) was added slowly at room temperature and the resulting solution was stirred at 50° C. for ˜4 hours. The solution was then cooled to ˜5° C. and water (4 mL) was added. Sodium hydroxide solution (25%, 14 mL) was added slowly to bring the pH to ˜13. Ethyl acetate (19 mL) was added and two layers were separated. The aqueous layer was extracted with ethyl acetate (2×10 mL). The combined organic layers were washed with water (4 mL), dried over sodium sulfate and then concentrated to minimum volume. Acetone (24 mL) was added and stirred until a clear solution was obtained. A solution of L-(+)-tartaric acid (0.75 g, 5.0 mmol, 1.0 eq) in water (2.0 mL) was added under stirring. The suspension was then stirred for 1 hour at room temperature before isolation. The solid was washed with acetone (2 mL) and then dried under house vacuum at 50° C. Example 10 as a white solid (2.4 g) was obtained.

### Example 11

### Preparation of trans-3-aminocyclobutyl (1S,2R,3S,4S,6R,7R,8R,14R)-4-ethenyl-3-hydroxy-2,4,7,14-tetramethyl-9-oxotricyclo[5.4.3.01,8]tetradec-6-yl imidodicarbonate L-tartrate

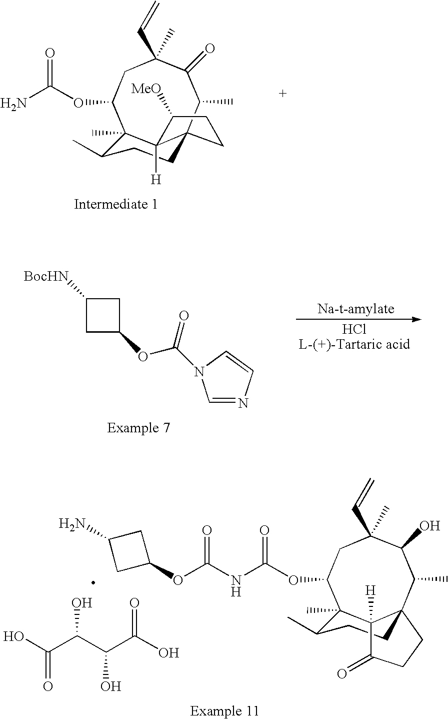

A solution of Intermediate 1 (807 g) in THF (4.0 L) was cooled to ˜0° C. in a 20 L jacketed laboratory reactor. Sodium tert-pentoxide (587 g) was added portionwise over 7 minutes to maintain the reaction temperature below 10° C. The solution was warmed to ˜15° C. over ˜15 minutes, then stirred at this temperature for ˜70 minutes, before cooling the solution back down to approximately ˜5 to 0° C. A solution of intermediate Example 7 (600 g) in THF (4.0 L) was added slowly over 25 minutes at a rate sufficient to keep the reaction temperature below 5° C. The solution was stirred for 2 hours. Water (3.2 L) was added slowly to quench the reaction while keeping the reaction temperature at ˜18° C. The solution was concentrated under vacuum until ˜6.5 L of solution remained. Dichloromethane (6.0 L) was added and the resulting two layers were separated and the aqueous layer was extracted twice with dichloromethane (3.0 L each). The combined organic layers were washed with water (1.7 L) then the organic layer was concentrated until ˜2 L of solution remained. THF (2.8 L) was added and the solution was kept at ambient conditions for ˜2.5 days.

The solution was cooled to ˜5° C. and concentrated hydrochloric acid (3.4 L) was added slowly while maintaining the solution temperature at <25° C. The resulting mixture was warmed to 34° C. over 20 minutes and stirred at ˜35° C. for ˜2.5 hours at which point the reaction was deemed complete by HPLC. The solution was then cooled to ˜5° C. over ˜20 minutes and water (1.6 L) was added. Sodium hydroxide solution (25%) was added slowly to bring pH to ˜9.0. The mixture was concentrated under vacuum at ˜15° C. Dichloromethane (6.0 L) was added and the two layers were separated. The aqueous layer was extracted with dichloromethane (3.0 L then 2.0 L). The combined organic layers were washed with brine (2.0 L) and then water (2.0 L) then concentrated to a final volume of ˜2.2 L then held overnight. Acetonitrile (9.6 L) was added and the solution was concentrated under vacuum until ˜12 L remained. Water (580 mL) was added and the mixture was heated to ˜50-55° C. before a solution of L-(+)-tartaric acid (319 g) in water (0.58 L) was added portionwise under stirring. The solution was then stirred for 1 hour at 55° C., a thick slurry being formed, and then cooled down to ˜15° C. over 55 minutes before isolation by filtration. The solid was washed with acetonitrile (1.6 L) and then dried under house vacuum at 50° C. Example 11 as a white solid (966 g) was obtained in a yield of 70%.

### Example 12

### Recrystallization of trans-3-aminocyclobutyl (1S,2R,3S,4S,6R,7R,5R,14R)-4-ethenyl-3-hydroxy-2,4,7,14-tetramethyl-9-oxotricyclo[5.4.3.01,8]tetradec-6-yl imidodicarbonate L-tartrate

A 1000 mL jacketed reactor was charged with 50 g of crude trans-3-aminocyclobutyl (1S,2R,3S,4S,6R,7R,8R,14R)-4-ethenyl-3-hydroxy-2,4,7,14-tetramethyl-9-oxotricyclo[5.4.3.01,8]tetradec-6-yl imidodicarbonate L-tartrate. Acetonitrile (150 mL) and water (62.5 mL) were charged. The slurry was stirred and heated to ˜70° C. to give a clear solution. When dissolved the solution was cooled to 65° C. over about 15 minutes, and held for about 1 hour. Ground seed crystals (0.1 w/w %) were added (50 mg in 5 mL acetonitrile). The resulting suspension was stirred for 30 minutes at 65° C. and then cooled to 50° C. over about 20 minutes. Maintaining the temperature at 48-50° C., 55 mL of acetonitrile was charged every 15 minutes for 2 hours. The slurry was cooled to 0° C. over 1.5 hours. The cold slurry was stirred for 15 hours, and the solid was then isolated by filtration under pressure. The reactor and cake were washed with acetonitrile (200 mL). The cake was dried under briefly under nitrogen pressure and heated to 50-55° C. under vacuum for 3 hours. The procedure gave 46.2 g of white solid, 92% by weight.

### Example 13

### Preparation of trans-4-aminocyclohexyl (1S,2R,3S,4S,6R,7R,8R,14R)-4-ethenyl-3-hydroxy-2,4,7,14-tetramethyl-9-oxotricyclo[5.4.3.01,8]tetradec-6-yl imidodicarbonate hydrochloride

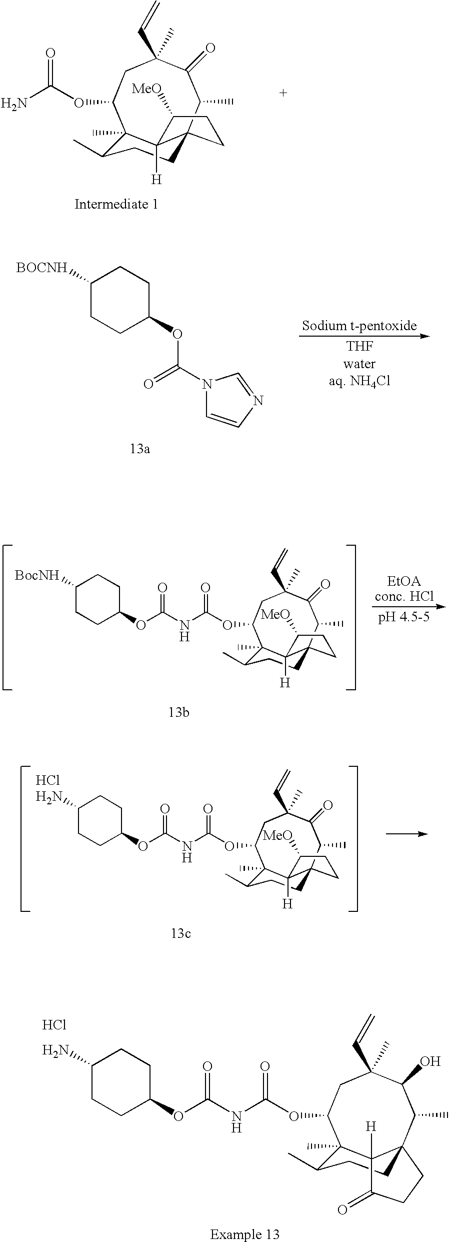

A solution of Intermediate 1 (5.0 g, 13.2 mmol, 1 eq) in dry THF (50 mL, 10 vols) under a nitrogen atmosphere was cooled to −5 to 0° C. To the solution was added sodium tert-pentoxide (3.6 g, 32.7 mmol, 2.5 eq) over ˜10 minutes, maintaining the process temperature below 5° C. The reaction mixture was stirred at 0 to 5° C. for ˜0.5 hour. To the reaction mixture was added 13a (4.5 g, 14.5 mmol, 1.1 eq) over ˜10 minutes, maintaining the process temperature below 5° C. The reaction mixture was stirred at ˜5° C. and after 1 hour aqueous NH₄Cl solution (70 mL, 14 vols, 7.5% w/w) was added slowly at 25° C. followed by ethyl acetate (60 mL, 12 vols). After stirring for ˜15 minutes the organic layer was separated and washed with aqueous NH₄Cl solution (35 mL, 7 vols, 15% w/w). The organic portion was concentrated to ˜8 volumes keeping the process temperature below ˜45° C. The crude product 13b in ethyl acetate was used in the next step directly.

The crude mixture was cooled to ˜10° C. and concentrated hydrochloric acid (20 mL, 4 vols) added slowly keeping the process temperature below 30° C. The reaction mixture was heated to ˜43° C. and the reaction progress monitored by HPLC. After 2 hours, the reaction was complete. The reaction mixture was cooled to ˜0° C. and water (20 mL, 4 vols) added, keeping the process temperature at ˜5° C. The pH of the reaction solution was adjusted to 4.5-5 by slowly adding aqueous ammonium hydroxide (˜30% solution, ˜15 mL, ˜3 vols) over 20 minutes keeping the process temperature below 15° C. The reaction mixture/suspension was cooled to ˜0° C., stirred for ˜1 hour and filtered. The wet cake was washed with ethyl acetate (25 mL, 5 vols) twice. The solid product was dried under vacuum to constant weight at ˜20° C. (LOD ˜5.2% w/v). Weight of solid product was 7.2 g (72% w/w).

Note: All charges were based on the amount of Intermediate 1.

### Example 14

### Preparation of trans-4-aminocyclohexyl (1S,2R,3S,4S,6R,7R,8R,14R)-4-ethenyl-3-hydroxy-2,4,7,14-tetramethyl-9-oxotricyclo[5.4.3.01,8]tetradec-6-yl imidodicarbonate succinate

Into an extraction funnel was added 16 mL of dichloromethane (“DCM”), 4 g of crude Example 13 (net 2.2 g), and 1.35 g of potassium carbonate dissolved in 12 mL of water. The layers were mixed well, and the dichloromethane layer was removed. The aqueous layer was re-extracted with 8 mL of dichloromethane. The dichloromethane layers were combined and extracted with 8 mL of water.

The dichloromethane was divided into two ˜equal portions and filtered into flesh flasks. To one portion was added 0.24 g succinic acid (1.0 eq) that was dissolved in 5 mL of n-propanol. The solution was seeded with trans-4-aminocyclohexyl (1S,2R,3S,4S,6R,7R,5R,14R)-4-ethenyl-3-hydroxy-2,4,7,14-tetramethyl-9-oxotricyclo[5.4.3.01,8]tetradec-6-yl imidodicarbonate succinate and distilled at atmospheric pressure to remove the dichloromethane. The solution was then cooled to room temperature and the product was isolated by filtration and dried to give 82% yield.

The total succinic acid content of the product was measured upon dissolution by HPLC and determined to be 1.0 molar equivalents. In addition, solid-state NMR of the product confirmed the presence of the core solid-state structure of trans-4-aminocyclohexyl (1S,2R,3S,4S,6R,7R,8R,14R)-4-ethenyl-3-hydroxy-2,4,7,14-tetramethyl-9-oxotricyclo[5.4.3.01,8]tetradec-6-yl imidodicarbonate succinate and additional succinic acid. The additional succinic acid content is 0.5 molar equivalents.

### Example 15

### Preparation of trans-4-aminocyclohexyl (1S,2R,3S,4S,6R,7R,8R,14R)-4-ethenyl-3-hydroxy-2,4,7,14-tetramethyl-9-oxotricyclo[5.4.3.01,8]tetradec-6-yl imidodicarbonate succinate

Into an extraction funnel was added 8 mL of dichloromethane (DCM), 2 g of crude Example 13 (net 1.34 g), and 0.675 g of potassium carbonate dissolved in 6 mL of water. The layers were mixed well, and the dichloromethane layer was removed. The aqueous layer was re-extracted with 4 mL of dichloromethane. The dichloromethane layers were combined and extracted with 4 mL of water.

0.35 g of succinic acid (1.2 eq) was dissolved in 12 mL of n-propanol and added to the dichloromethane solution. The solution was stirred and seeded with trans-4-aminocyclohexyl (1S,2R,3S,4S,6R,7R,5R,14R)-4-ethenyl-3-hydroxy-2,4,7,14-tetramethyl-9-oxotricyclo[5.4.3.01,8]tetradec-6-yl imidodicarbonate succinate and left overnight. The slurry was distilled at atmospheric pressure to remove the dichloromethane. The solution was then cooled to room temperature and the product was isolated by filtration and dried to give 89% yield.

The total succinic acid content of the product was measured upon dissolution by HPLC and determined to be 1.0 molar equivalents. In addition, solid-state NMR of the product confirmed the presence of the core solid-state structure of trans-4-aminocyclohexyl (1S,2R,3S,4S,6R,7R,8R,14R)-4-ethenyl-3-hydroxy-2,4,7,14-tetramethyl-9-oxotricyclo[5.4.3.01,8]tetradec-6-yl imidodicarbonate succinate and additional succinic acid. The additional succinic acid content is 0.5 molar equivalents.

### Example 16

### Preparation of trans-4-aminocyclohexyl (1S,2R,3S,4S,6R,7R,5R,14R)-4-ethenyl-3-hydroxy-2,4,7,14-tetramethyl-9-oxotricyclo[5.4.3.01,8]tetradec-6-yl imidodicarbonate succinate

Into an Erlenmeyer flask was added 56 mL of dichloromethane (DCM), 14 g of crude Example 13 (net 9.4 g), and 4.72 g of potassium carbonate dissolved in 42 mL of water. The layers were mixed well and transferred to a separatory fennel. The dichloromethane layer was removed. The aqueous layer was re-extracted with 28 mL of methylene chloride. The dichloromethane layers were combined and extracted with 28 mL of water.

2.46 g of succinic acid (1.2 eq) was dissolved in 84 mL of n-propanol and added to the dichloromethane solution. The solution was seeded with trans-4-aminocyclohexyl (1S,2R,3S,4S,6R,7R,5R,14R)-4-ethenyl-3-hydroxy-2,4,7,14-tetramethyl-9-oxotricyclo[5.4.3.01,8]tetradec-6-yl imidodicarbonate succinate. The slurry was distilled at atmospheric pressure over 4 h to remove the dichloromethane. The solution was then cooled to room temperature overnight and cooled to 0° C. and the product was isolated by filtration and dried to give 86% yield, 9.29 gm.

### Example 17

### Preparation of trans-4-aminocyclohexyl (1S,2R,3S,4S,6R,7R,5R,14R)-4-ethenyl-3-hydroxy-2,4,7,14-tetramethyl-9-oxotricyclo[5.4.3.01,8]tetradec-6-yl imidodicarbonate succinate

A 1 L jacketed reactor was charged with 240 mL of dichloromethane (DCM) and 60 g of crude Example 13 (net 38.7 g). Potassium carbonate (20.2 g) was dissolved in 180 mL of water and charged to the reactor over 15 minutes. The reaction was stirred for 15 minutes, settled, and then the layers were separated. The aqueous layer was re-extracted with 120 mL of dichloromethane. The dichloromethane layers were combined and extracted with 120 mL of water. The dichloromethane layers were filtered into a 1 L flask.

10.14 g of succinic acid (1.2 eq) was dissolved in 300 mL of n-propanol and added to the dichloromethane solution. The solution was seeded with trans-4-aminocyclohexyl (1S,2R,3S,4S,6R,7R,5R,14R)-4-ethenyl-3-hydroxy-2,4,7,14-tetramethyl-9-oxotricyclo[5.4.3.01,8]tetradec-6-yl imidodicarbonate succinate The slurry was distilled at atmospheric pressure over ˜5 h to remove the dichloromethane and stirred at room temperature overnight. The solution was then slowly cooled to 5° C. in an ice bath and the product was isolated by filtration and washed with 180 mL of cold n-propanol. The wet cake was dried under vacuum at 60° C. to give 84% yield, 37.2 g.

### Example 18

### Preparation of trans-4-aminocyclohexyl (1S,2R,3S,4S,6R,7R,8R,14R)-4-ethenyl-3-hydroxy-2,4,7,14-tetramethyl-9-oxotricyclo[5.4.3.01,8]tetradec-6-yl imidodicarbonate edisylate

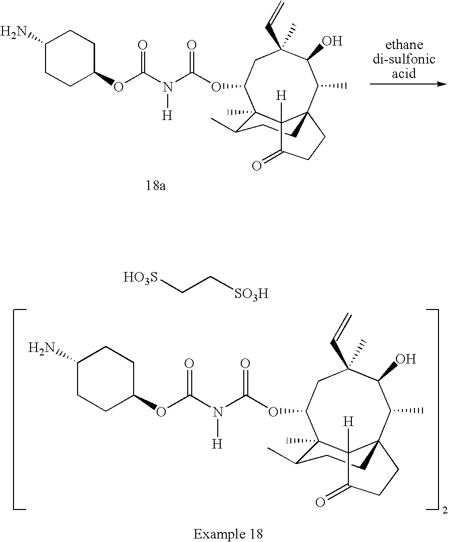

To a 20-mL test tube, trans-4-aminocyclohexyl (1S,2R,3S,4S,6R,7R,8R,14R)-4-ethenyl-3-hydroxy-2,4,7,14-tetramethyl-9-oxotricyclo[5.4.3.01,8]tetradec-6-yl imidodicarbonate (18a) (150 mg) was added, followed by 8 ml of a mixture of EtOAc and MeOH (17 to 1) at room temperature. The mixture was carefully warmed up to about 40-45° C. to give a clear solution. To the solution, a 1N aqueous solution of 1,2-ethanedisulfonic acid (0.297 mL) was added. Within 30 minutes, the contents in the test tube turned to off-white slurry. The slurry was stirred over 24 hours under a condition that the temperature was oscillated between 10 and 50° C. After cooling the contents to room temperature, the solid was isolated by filtration under vacuum, and was washed by hexane (5 mL). The solid was dried in a vacuum oven at 20-25° C. over 16 hours.

### Example 19

### Preparation of trans-4-aminocyclohexyl (1S,2R,3S,4S,6R,7R,8R,14R)-4-ethenyl-3-hydroxy-2,4,7,14-tetramethyl-9-oxotricyclo[5.4.3.01,8]tetradec-6-yl imidodicarbonate edisylate

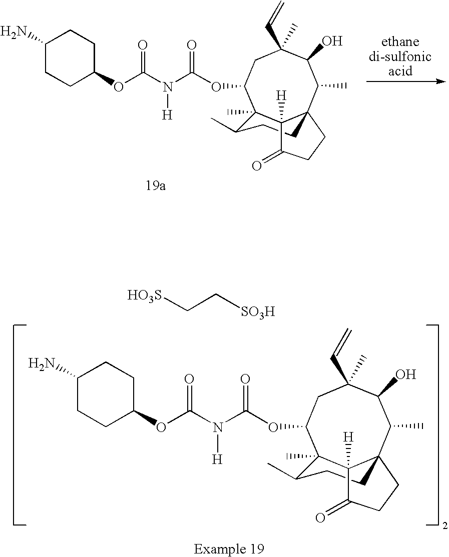

To a 500-mL round bottom flask was added trans-4-aminocyclohexyl (1S,2R,3S,4S,6R,7R,8R,14R)-4-ethenyl-3-hydroxy-2,4,7,14-tetramethyl-9-oxotricyclo[5.4.3.01,8]tetradec-6-yl imidodicarbonate (19a) (12.01 g, 23.78 mmole; PL204670-184), followed by THF (25 mL) and EtOAc (220 mL) at room temperature. The mixture was carefully warmed up to about 50-52° C. to gave a clear solution. To a 50-mL round bottom flask, 1,2-ethanedisulfonic acid (2.26 g, 11.89 mmole) was added, followed by in THF (10 mL) and water (3 mL). The mixture was warmed to about 30-35° C., and a clear solution was seen. To the clear solution of SB 742510, the solution of 1,2-ethanedisulfonic acid was added slowly at 45-50° C. over 20 minutes. In the middle of this addition, another 10 mL of THF and 50 mL of EtOAc were added to improve the mixing of the contents in the flask. After the addition of acid solution, the reaction contents were cooled to 20-25° C., and held for one hour. The salt was isolated by filtration and washed with mixture of EtOAc and cyclohexane (50/50, 20 mL). The crude salt was dried in a vacuum oven at 45-50° C. over 16 hours to give a final weight of 12.08 g.

To a 500-mL flask was added the crude edisylate salt (10.9 g) followed by THF (33 mL), EtOAc (126 mL) and MeOH (20 mL) at room temperature. The mixture was warmed to 45-50° C. and it was still a white slurry. To the slurry a small amount (about 50 mg) of authentic edisylate salt was added as seed, and it was stirred over 17 hours at 38-40° C. The slurry was cooled to 20-22° C. and filtered to give a white cake, which was washed by THF (20 mL). The cake was dried in a vacuum oven at 45-50° C. over 16 hours to give a final weight of 10 g.

1. A compound according to Formula I: 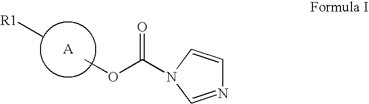
wherein:
A is C4-C6 cycloalkyl, and
R1 is a protected amine or an amine precursor.

2. A compound according to claim 1 wherein A is cyclobutyl.

3. A compound according to claim 1 wherein A is cyclohexyl.

4. A compound according to claim 1 wherein R1 is a protected amine.

5. A compound according to claim 4 wherein the protected amine is substituted with one suitable protecting group.

6. A compound according to claim 5 wherein the suitable protecting group is —C(O)Z1 wherein Z1 is O-alkyl, O-alkenyl, O-cycloalkyl, O-cycloalkenyl, O-heterocycloalkyl, O-aryl, or O-heteroaryl.

7. A compound according to claim 5 wherein the suitable protecting group is —C(O)Z2 wherein Z2 is H, alkyl, alkenyl, cycloalkyl, cycloalkenyl, heterocycloalkyl, aryl, or heteroaryl.

8. A compound according to claim 5 wherein the suitable protecting group is —S(O₂)X1 wherein X1 is alkyl, alkenyl, cycloalkyl, cycloalkenyl, heterocycloalkyl, aryl, or heteroaryl.

9. A compound according to claim 5 wherein the suitable protecting group is —S(O)X2 wherein X2 is alkyl, alkenyl, cycloalkyl, cycloalkenyl, heterocycloalkyl, aryl, or heteroaryl.

10. A compound according to claim 5 wherein the suitable protecting group is —N(Rx)(Ry) wherein Rx and Ry taken together with the nitrogen atom to which they are attached form an optionally substituted succinimide ring, an optionally substituted maleimide ring, or an optionally substituted phthalimide ring.

11. A method of preparing a compound having the following general structure: 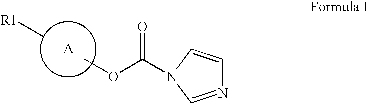
wherein:
A is C4-C6 cycloalkyl, and
R1 is a protected amine or an amine precursor comprising the steps of:
a) providing a compound having the following general structure: 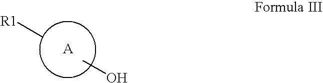
wherein R1 and A are as defined above for Formula I; and
b) reacting the compound according to Formula III with 1,1′-carbonyldiimidazole to yield a compound according to Formula I.
